SEQUENCE LISTING (15 sequences)

SEQ ID NO: 1 - DNA, 1011 bp, Artificial sequence
  Chloroplast codon usage optimized ptxD nucleotide sequence
atgttaccaaaattagttattacacatcgtgttcatgatgaaattttacaattattagctccacattgtgaattaatgacaaatcaaacagattcaacattaacacgtgaagaaattttacgtcgttgtcgtgatgctcaagctatgatggcttttatgccagatcgtgttgatgctgattttttacaagcttgtccagaattacgtgttgttggttgtgctttaaaaggttttgataattttgatgttgatgcttgtacagctcgtggtgtttggttaacatttgttccagatttattaacagttccaacagctgaattagctattggtttagctgttggtttaggtcgtcatttacgtgctgctgatgcttttgttcgttcaggtgaatttcaaggttggcaaccacaattttatggtacaggtttagataatgctacagttggtattttaggtatgggtgctattggtttagctatggctgatcgtttacaaggttggggtgctacattacaatatcatgaagctaaagctttagatacacaaacagaacaacgtttaggtttacgtcaagttgcttgttcagaattatttgcttcatcagattttattttattagctttaccattaaatgctgatacacaacatttagttaatgctgaattattagctttagttcgtccaggtgctttattagttaatccatgtcgtggttcagttgttgatgaagctgctgttttagctgctttagaacgtggtcaattaggtggttatgctgctgatgtttttgaaatggaagattgggctcgtgctgatcgtccacgtttaattgatccagctttattagctcatccaaatacattatttacaccacatattggttcagctgttcgtgctgttcgtttagaaattgaacgttgtgctgctcaaaatattattcaagttttagctggtgctcgtccaattaatgctgctaatcgtttaccaaaagctgaaccagctgcttgttaa

SEQ ID NO: 2 - DNA, 60 bp, Artificial sequence
  Mismatch forward primer
ttgttctgtttgtgtatctaaagctttacgagcatgatattgtaatgtagcaccccaacc

SEQ ID NO: 3 - DNA, 60 bp, Artificial sequence
  Mismatch reverse primer
ggttggggtgctacattacaatatcatgctcgtaaagctttagatacacaaacagaacaa

SEQ ID NO: 4 - DNA, 20 bp, Artificial sequence
  ptxDamplification FW primer
cctcgtccgacttcatcctg

SEQ ID NO: 5 - DNA, 20 bp, Artificial sequence
  ptxD amplification RV primer
cccagtcctccatctcgaag

SEQ ID NO: 6 - DNA, 25 bp, Artificial sequence
  Chloroplast codon usage ptxD amplification FW primer
ccaaaattagttattacacatcgtg

SEQ ID NO: 7 - DNA, 23 bp, Artificial sequence
  Chloroplast codon usage ptxDamplification RV primer
catgatattgtaatgtagcaccc

SEQ ID NO: 8 - DNA, 25 bp, Artificial seqeunce
  ATPase beta subunit amplification FW primer
gtaaatacttcagctacgaagaatg

SEQ ID NO: 9 - DNA, 27 bp, Artificial sequence
  ATPase beta subunit amplification RV primer
atgttaacaaacaagacgtattattct

SEQ ID NO: 10 - protein, 14 aa, Artificial sequence
  GS peptide linker
GGGGSSGGGGGGSS

SEQ ID NO: 11 - DNA, 1991 bp, Artificial sequence
  Chimeric construct comprising the optimized ptxD version
ccatggctgaagttgttttaacagatattggtgctacagatattacatttaaaggttttccagttacaatggaattaaatttttggaatgttaaatcatatgaaggtgaaacatggttaaaatttgatggtgaaaaagttcaattttatgctgatatttataatattgttttacaaaatccagattcatgggttcatggttatccagaaatttattatggttataaaccttgggctgctcataattcaggtacagaaattttaccagttaaagttaaagatttaccagatttttatgttacattagattattcaatttggtatgaaaatgatttaccaattaatttagctatggaaacatggattacacgtaaaccagatcaaacatcagtttcatcaggtgatgttgaaattatggtttggttttataataatattttaatgccaggtggtcaaaaagttgatgaatttacaacaacaattgaaattaatggttcaccagttgaaacaaaatgggatgtttattttgctccttggggttgggattatttagcttttcgtttaacaacaccaatgaaagatggtcgtgttaaatttaatgttaaagattttgttgaaaaagctgctgaagttattaaaaaacattcaacacgtgttgaaaattttgatgaaatgtatttttgtgtttgggaaattggtacagaatttggtgatccaaatacaacagctgctaaatttggttggacatttaaagatttttcagttgaaattggtgaaggtggtggtggttcatcaggtggtggtggtggtggttcatcaatggctatggctatgcgttcaacatttgctgctcgtgttggtgctaaaccagctgttcgtggtgctcgtccagcttcacgtatgtcatgtatggctatggattataaagatcatgatgattataaagatcatgatgattataaagatgatgatgataaattaccaaaattagttattacacatcgtgttcatgatgaaattttacaattattagctccacattgtgaattaatgacaaatcaaacagattcaacattaacacgtgaagaaattttacgtcgttgtcgtgatgctcaagctatgatggcttttatgccagatcgtgttgatgctgattttttacaagcttgtccagaattacgtgttgttggttgtgctttaaaaggttttgataattttgatgttgatgcttgtacagctcgtggtgtttggttaacatttgttccagatttattaacagttccaacagctgaattagctattggtttagctgttggtttaggtcgtcatttacgtgctgctgatgcttttgttcgttcaggtgaatttcaaggttggcaaccacaattttatggtacaggtttagataatgctacagttggtattttaggtatgggtgctattggtttagctatggctgatcgtttacaaggttggggtgctacattacaatatcatgctcgtaaagctttagatacacaaacagaacaacgtttaggtttacgtcaagttgcttgttcagaattatttgcttcatcagattttattttattagctttaccattaaatgctgatacacaacatttagttaatgctgaattattagctttagttcgtccaggtgctttattagttaatccatgtcgtggttcagttgttgatgaagctgctgttttagctgctttagaacgtggtcaattaggtggttatgctgctgatgtttttgaaatggaagattgggctcgtgctgatcgtccacgtttaattgatccagctttattagctcatccaaatacattatttacaccacatattggttcagctgttcgtgctgttcgtttagaaattgaacgttgtgctgctcaaaatattattcaagttttagctggtgctcgtccaattaatgctgctaatcgtttaccaaaagctgaaccagctgcttgttaagcatgc

SEQ ID NO: 12 - DNA, 25 bp, Artificial sequence
  CelB amplification FW primer
gctgaagttgttttaacagatattg

SEQ ID NO: 13 - DNA, 23 bp, Artificial sequence
  CelB amplification RV primer
caaccccaaggagcaaaataaac

SEQ ID NO: 14 - DNA, 21 bp, Artificial sequence
  psbD amplification FW primer
gagcgttaccacgtggtaata

SEQ ID NO: 15 - DNA, 21 bp, Artificial sequence
  psbD amplification RV primer
gttctttgcacctagtttcgg

(19) United States
(12) Patent Application Publication
(10) Pub. No.: US 2024/0392258 A1
(43) Pub. Date: Nov. 28, 2024

(54) GENETIC STABILIZER AND GROWTH SELECTOR FOR GENETIC TRANSFORMATION OF THE CHLOROPLAST GENOME OF MICROALGAE

(71) Applicants: Edoado Andrea CUTOLO, Genova (IT); Roberto BASSI, Verona (IT); Luca DALL'OSTO, San Martino Buon Albergo (VR) (IT)

(72) Inventors: Edoardo Andrea CUTOLO, Genova (IT); Roberto BASSI, Verona (IT); Luca DALL'OSTO, San Martino Buon Albergo (VR) (IT)

(21) Appl. No.: 18/279,470

(22) PCT Filed: Feb. 1, 2021
(86) PCT No.: PCT/IB2021/050785

§ 371 Date: Aug. 30, 2023

(51) Int. Cl. C12N 9/02; C12N 1/12; C12N 15/82

(52) U.S. Cl. CPC C12N 9/0004; C12N 1/12; C12N 15/8216; C12N 15/8221; C12Y 120/01001

(57) ABSTRACT

The present invention relates to a modified ptxD nucleotide sequence and related uses as a genetic stabilizer and growth selector for the genetic transformation of the chloroplast genome of microalgae and methods for enhance the expression of recombinant proteins of interest in large-scale algal cultivation.

The present invention relates to a modified ptxD nucleotide sequence and related uses as a genetic stabilizer and growth selector for the genetic transformation of the chloroplast genome of microalgae and methods for enhance the expression of recombinant proteins of interest in large-scale algal cultivation.

Chloroplast transformation (transplastomic technology) represents a powerful tool to introduce transgenes into the genome of the photosynthetic semi-autonomous organelle.

Microalgae hold a tremendous potential for becoming the preferential host species for the production of valuable bioactive compounds and recombinant proteins directly in the plastid [1]. The single chloroplast of the alga represents its largest subcellular compartment and an ideal storage site for recombinant products.

The major advantages offered by microalgae, such as Chlamydomonas reinhardtii, as a biofactory lie in its unicellular, photoautotrophic lifestyle and the ease of transforming its chloroplast genome via homologous recombination-based approaches [2]. Being the plastid genome polyploid [3], multiple transgene copies are present in a transplastomic algal cell, boosting yield in recombinant proteins.

In contrast to nuclear transgenesis, where DNA insertion occurs randomly in the genome [4], sequences can be targeted to defined chloroplast loci [5]. Moreover, the plastid genome does not seem to be subjected to epigenetic effects and, therefore, does not entail silencing on transgene expression, as it occurs in the nucleus [6].

Several obstacles, however, currently hinder the industrial use of microalgae. The persistence of antibiotic resistance genes (ARGs) in algal genomes prevents market access due to health-related concerns over the risk of ARG horizontal transfer to harmful pathogens and their diffusion in the environment. Also, the release of cultivation waste waters containing large doses of antibiotics in the environment might promote the occurrence of resistance in some health-threatening organisms. Secondly, genetic manipulation of the polyploid plastome [3] is potentially unstable, making current expression systems unreliable.

Perhaps, the strongest challenge faced by large-scale algal cultivation is the ubiquitous risk of parasitic contamination by bacteria, oomycetes, outcompeting and fast-growing weedy algae, cyanobacteria and other biological pollutants, which can severely affect biomass production, both in closed photobioreactors and outdoor high-rate pond systems ([7, 8]).

Removal of these barriers will favor the industrial exploitation of microalgae for the sustainable production of recombinant proteins [9].

Given these premises, there is an urgent need to abandon the use of antibiotics in the field of transplastomic algal biotechnology and to foster the development of alternative selective growth strategies which do not involve the use of potentially toxic compounds.

A recent breakthrough in the field has been the introduction of a novel pest control system based on the nuclear expression of the ptxD transgene, encoding the NAD⁺-dependent phosphite (Phi) dehydrogenase from the soil bacterium Pseudomonas stutzeri WM88 ([10,11,12]). Chloroplast expression of ptxD in Chlamydomonas reinhardtii was proposed as an environmentally friendly alternative to antibiotic resistance genes for plastid transformation ([13,14]). Since Phi can only be assimilated in the metabolism following oxidative conversion into phosphate (Pi, PO₄³⁻) [15] by dedicated Phi-dehydrogenases, non-transgenic organism, including most parasites, are prevented from growing in Phi-fertilized media [12].

However, PTXD activity in the chloroplast is low, possibly due to the low NAD⁺/NADP⁺ ratio, limiting the efficiency of the selection method.

Previous in vitro works have sought to modify the enzyme by means of rational mutagenesis and direct evolution, mainly focusing on thermal stability [16] and cofactor binding properties [17]. Despite being an attractive system for the chloroplast transformation of C. reinhardtii, the use of PTXD as a selectable marker suffers from some intrinsic limitations that hinder its value. Indeed, the PTXD-based selection displays a far lower efficiency compared to traditional antibiotic resistance-based methods [14], mainly due to long selection time (reported up to 60 days), which reduces the recovery efficiency of true transformation events.

The authors of the present invention recently developed an optimized version of the PTXD enzyme which is mutagenized in two adjacent amino acids located in the cofactor-binding pocket of the enzyme with the aim of relaxing the PTXD selectivity towards the nicotinamide adenine cofactors, rendering the enzyme equally functional when using NAD⁺ or NADP⁺ and thus increasing its catalytic efficiency in vivo in the algal chloroplast stromal compartment [18].

Such optimized version of the PTXD enzyme expressed in the chloroplast (and not in the nucleus) bypassed the physiological limitations imposed by the chloroplast metabolism, showing efficient function in this predominantly NADP⁺-enriched environment.

Additionally, the authors of the present invention developed a ptxD/Phi platform for the genetic engineering of the algal chloroplast in the model species Chlamydomonas reinhardtii to produce a hydrolytic enzyme and ensuring genetic stability and reliable expression of the desired recombinant product. The peculiar construct design, consisting of two elements connected by an in vivo cleavable linker, leads to the synthesis of a hydrolase-PTXD chimera and its subsequent processing by a plastid resident protease. In this way, the selective pressure imposed by phosphite metabolism relying on the assimilatory oxidation exerted by PTXD prevents transgene rearrangements and/or loss favouring, at the same time, stable expression of the hydrolytic enzyme and algal cultivation in axenic, non-sterile conditions. This experimentally demonstrated proof of concept provides an example of genetic engineering in which biosafety is integrated in the sustainable management of large-scale microalgal monocultures for the production of enzymes, such as cellulose-degrading enzymes.

Therefore, it is an object of the present invention a ptxD nucleotide sequence encoding for the NAD⁺ dependent-phosphite dehydrogenase D (PTXD) enzyme of Pseudomonas stutzeri WM88 (Gene Bank AF061070.1) characterized by:
the substitution of the GAA codone encoding for glutamic acid 175 by a codone encoding for alanine residue;
the substitution of the GCT codone of alanine 176 by a codone encoding for arginine residue;
the optimization of each codon according to the AT-rich codon bias of the chloroplast genome of microalgae;
   wherein said nucleotide sequence is DNA or RNA sequence.

With the expression “optimized ptxD nucleotide sequence” as used in the foregoing disclosure it is intended a modified nucleotide sequence of ptxD gene carrying the above substitutions and codon usage optimization.

According to a preferred embodiment of the invention, said microalgae belong to the genus Chlamydomonas or Chlorella. According to a more preferred embodiment, said microalgae belongs to the species Chlamydomonas reinhardtii. In an alternative embodiment said microalgae belongs to the species Chlorella vulgaris, Chlorella sorokiniana or Chlorella ohadii. The chloroplast codon usage to be used for the transformation of Chlorella with the optimized ptxD sequence is the same of the one used in Chlamydomonas.

The wild-type PTXD enzyme of bacterial origin uses preferentially NAD⁺ as cofactor to convert phosphite into phosphate—a key nutrient for the growth and cell activities of all the organisms—but cannot efficiently use the phosphorylated cofactor pool, and thus only relies on the low-abundant NAD⁺ for the catalytic conversion of phosphite into phosphate. On the other hand, the NADP+ cofactor is more abundant in the chloroplast of microalgae. This cofactor is characterized by the presence of a negatively group, the phosphate, preventing the accommodation within the binding site of the cofactor present in the native PTXD enzyme.

The double amino acid substitution in the optimized version of the enzyme has the effect to modify the charge distribution in the binding site of the modified PTXD which displays an increased affinity for the native cofactor NAD⁺, as well as an even stronger increase in the affinity for the NADP⁺ cofactor, which is enriched in plastids.

Additionally, the catalytic efficiency of the mutagenized enzyme is significantly higher compared to the wild-type version as it displays a higher turn-over rate, which is reflected by a faster conversion rate of phosphite into phosphate and, ultimately, in the synthesis of phosphorylated sugar moieties, with a positive impact on the algal growth.

However, also the physiological conditions of the chloroplast should be taken into duly consideration and for this reason the wild-type sequence has been optimized for the preferential codon usage of the chloroplast genome of microalgae (particularly, rich of adenine and thymine). This optimization of the codons of the nucleotide sequences destined to chloroplast expression is necessary to carry out the protein synthesis inside the chloroplast depending on a transcription/translation apparatus having prokaryotic origin, which has different function with respect to the nuclear one.

According to a preferred embodiment of the invention, the ptxD nucleotide sequence comprises or consists of SEQ ID NO:1.

It is a further object of the present invention a plasmid or a vector comprising the optimized ptxD nucleotide sequence of the invention. Preferably, the optimized nucleotide sequence is placed under the transcriptional control of cis-acting elements.

The invention further relates to the use of the optimized ptxD nucleotide sequence, the plasmid or vector comprising such nucleotide sequence as a growth selector for the genetic transformation of the chloroplast genome of microalgae, cultivated in a phosphite-fertilized medium (as sole phosphorous source).

Another object of the invention relates to the use of the optimized ptxD nucleotide sequence as a genetic stabilizer to maintain and enhance the expression of recombinant proteins inside the chloroplast in large-scale microalgal cultivation.

It is another object of the invention a chimeric construct comprising the following elements:
i) restriction sites at 3′ and 5′ ends;
ii) the nucleotide sequence of the transgene encoding for a protein of interest;
iii) a linker;
iv) a transit peptide;
v) the optimized ptxD nucleotide sequence.

According to a preferred embodiment of the chimeric construct, the restriction sites at 3′ and 5′ ends are NcoI and SphI, respectively. According to another preferred embodiment the linker is a GS linker. According to another preferred embodiment the transit peptide used as in vivo cleavable substrate is a PreFd element, corresponding to the transit peptide of the preferredoxin precursor. In an alternative embodiment of the present invention said transit peptide may be selected from the group consisting of nucleus-encoded, chloroplast imported precursor proteins comprising, among others, the small subunit of the RuBisCo multienzyme complex, the pigment-binding Light-Harvesting Complex proteins of photosystems (LHCs) and Calvin Cycle enzymes involved in CO₂ fixation.

The position of the optimized ptxD nucleotide sequence in the chimeric construct is preferably downstream in order to promote the accumulation of the fully synthetized fusion polypeptide comprising the two protein elements, granting the Phi-metabolizing activity of the PTXD moiety. The expression of a truncated chimera would in fact compromise the selective growth phenotype and thus the genetic stability of the system.

In the most preferred embodiment of the invention the chimeric construct comprises or consists of SEQ ID NO:11.

A further object of the invention relates to the use of anyone of the chimeric construct above disclosed as genetic stabilizer and growth selector for the expression of recombinant proteins in microalgae cultivations, such as large scale microalgae cultivations. Preferably, said microalgae belong to the genus Chlamydomonas or Chlorella, even more preferably to the species Chlamydomonas reinhardtii, Chlorella vulgaris, Chlorella sorokiniana or Chlorella ohadii.

According to a preferred embodiment of the present invention said protein of interest is an hydrolase, preferably a cellulase. More preferably, said protein is Cel B endoglucanase from the hyperthermophilic bacterium Thermotoga neapolitana.

According to a further aspect, the present invention relates to an eukaryotic or prokaryotic cell transformed with the optimized ptxD nucleotide sequence, the plasmid, the vector or the chimeric construct as above defined. Preferably, said eukaryotic cell is microalgae, preferably belonging to the genus Chlamydomonas or Chlorella and even more preferably selected from the group of species comprising Chlamydomonas reinhardtii, Chlorella vulgaris, Chlorella sorokiniana and Chlorella ohadii.

The invention is further directed to the use of the transformed eukaryotic cell of Chlamydomonas, preferably of Chlamydomonas reinhardtii, for the large-scale cultivation and production of recombinant proteins characterized in that it is carried out in phosphite-fertilized medium. Said cultivation is preferably open-air cultivation or closed photobioreactors in non-sterile conditions. Fertilization with phosphite acts as a low-cost selective agent which forces the maintenance of the bifunctional transgene in the algal plastome, being the only phosphorous source available for the algal metabolism and whose incorporation strictly depends on the activity of the PTXD enzyme. Alternatively said transformed eukaryotic cell belongs to the genus Chlorella, more preferably to the species Chlorella vulgaris, Chlorella sorokiniana or Chlorella ohadii.

The present invention will now be described, for non-limiting illustrative purposes, according to a preferred embodiment thereof, with particular reference to the attached figures, wherein:

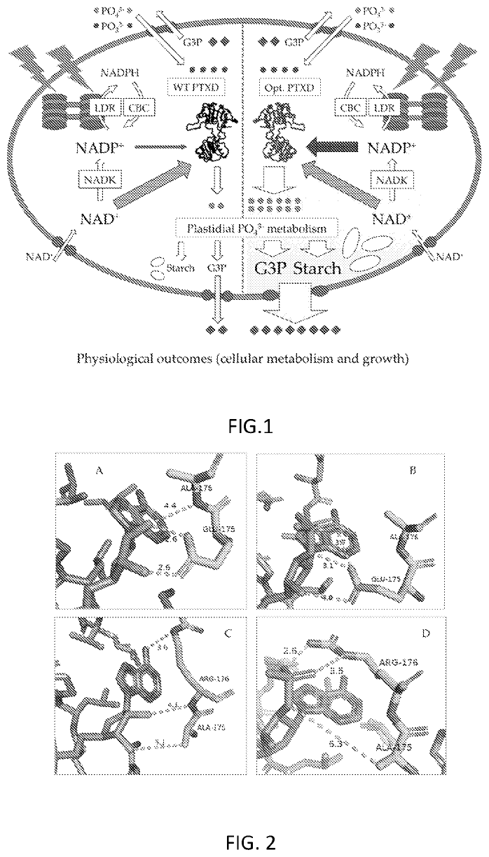

FIG. 1 shows a working model describing the effects of the optimized PTXD catalytic efficiency in the chloroplast of Chlamydomonas reinhardtii. Phosphate (PO₄³⁻, dark grey circles) and phosphite (PO₃³⁻, light grey circles) can be handled by the same type of transporters in biological systems and are thus imported into the chloroplast via the identical antiporter system in exchange with triose phosphates (G3P). The supply of NAD⁺ to the chloroplast relies on its unidirectional import from the cytoplasm, where it is converted in the phosphorylated form NADP⁺ by the calcium-dependent NAD⁺ kinase (NADK). NADP⁺ is the terminal acceptor of the linear electron transport chain of light-dependent reactions (LDR) of photosynthesis occurring on the thylakoid membranes (cylinders), being reduced by the soluble enzyme ferredoxin to NADPH. NADPH is then used in the Calvin-Benson cycle (CBC) to reduce CO₂ into sugars and, thus, undergoes a dynamic redox interconversion to regenerate NADP⁺. On the left side of the picture, the wild type (WT) PTXD enzyme uses it preferentially as cofactor NAD⁺ to convert phosphite into phosphate [10], which is then assimilated by the plastid metabolism to produce triose phosphates (G3P, rhombi) that are exported to the cytoplasm and/or are used for the in situ synthesis of starch (white ovals). The wild type PTXD cannot efficiently use the phosphorylated cofactor pool, and only relies on the low-abundant NAD⁺ for the catalytic conversion of phosphite into phosphate. On the right side of the picture, the modified enzyme (Opt. PTXD) carrying two amino acid residue substitutions within the cofactor binding pocket displays an increased affinity for the native cofactor NAD⁺, as well as an even stronger increase in the affinity for the NADP⁺ cofactor, which is enriched in plastids. Arrow sizes reflect the affinity of each PTXD version for the two cofactors (NAD⁺ and NADP⁺) and their catalytic output in terms of accumulated products triose phosphates and starch (downward pointing white arrows).

FIG. 2 shows a molecular model of the cofactor-binding pocket in the PTXD enzyme showing the effects of the double amino acid substitution (E175A, A176R) on the docking of the NAD⁺ vs NADP⁺ cofactors. The interactions between the nucleotide moiety of both cofactors with residues 175 and 176 are shown. Charge interactions are depicted as dashed lines with distances in Angstrom between the hydrogen bond acceptor atoms to hydrogen atoms. (A) NAD⁺ cofactor binding by the wild type enzyme, where multiple polar interactions are allowed between the 2′- and 3′-hydroxyl groups of the nucleotide and the side chain of Glutamate-175 and the peptidyl nitrogen of residue Alanine-176. (B) NADP⁺ binding by the wild type enzyme. This is a suboptimal interaction due to charge repulsion/steric hindrance effect from the negatively charged carboxyl group of Glutamate-175, which prevents docking. This is consistent with the observed lower K_{M} for NADP⁺. (C) Binding of NAD⁺ by the mutagenized PTXD. Interactions between cofactor and protein are similar to the case of WT since following the replacement of residue 175 with the smaller side chain of Alanine the distances are only slightly wider allowing for a sterical relaxation of the local charge environment. (D) Binding of NADP⁺ to the mutagenized enzyme. The substitution of Glutamate-175 to Alanine enables docking of the 2′-phosphate cofactor group, otherwise physically constrained. Moreover, the Alanine-176 to Arginine replacement results in a stabilization of the phosphate moiety of the cofactor via compatible ionic and polar interactions. Structural analysis was performed with PyMol software.

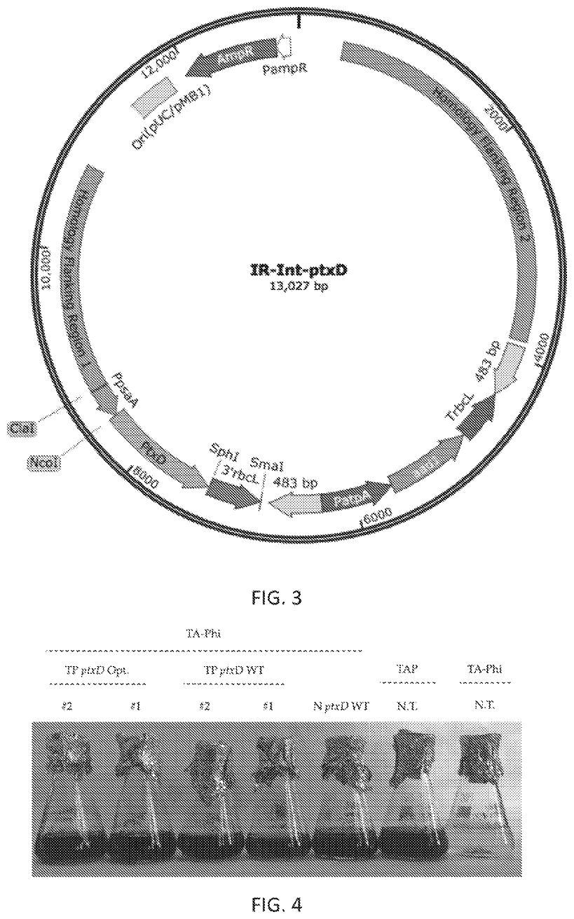

FIG. 3 shows the map of the assembled IR-Int vector carrying the ptxD gene that was used to transform the plastome of the wild type T222 strain. This vector contains two flanking regions required to enable the homologous-recombination based transgene insertion at the level of the inverted repeats of the chloroplast genome of the alga and the recyclable selectable marker gene aadA, providing resistance against the antibiotic spectinomycin. The PTXD gene was optimized according to the AT-rich codon bias of the chloroplast genome of C. reinhardtii and placed under the transcriptional control of the cis-acting elements pair psaA promoter and rbcL 3′ UTR and terminator via a subcloning step through the AtpB-int vector. A mutagenized version of the PTXD enzyme, carrying the substitution of the two adjacent residues Glu-175 and Ala-176 with Ala and Arg, respectively, was produced via site-directed mutagenesis and introduced in the same vector configuration (referred to as ptxD_{E175A-A176R}).

FIG. 4 shows the growth of selected transformant lines in TA-Phi (phosphite-fertilized) medium. Two independent transplatomic lines carrying the wild type PTXD (TP ptxD WT) and mutagenized (TP ptxD Opt) versions, respectively, were included, along with the nuclear transformant (N ptxD WT) and the untransformed T222 wild type. This latter was also cultivated in TAP (phosphate-fertilized) as positive control.

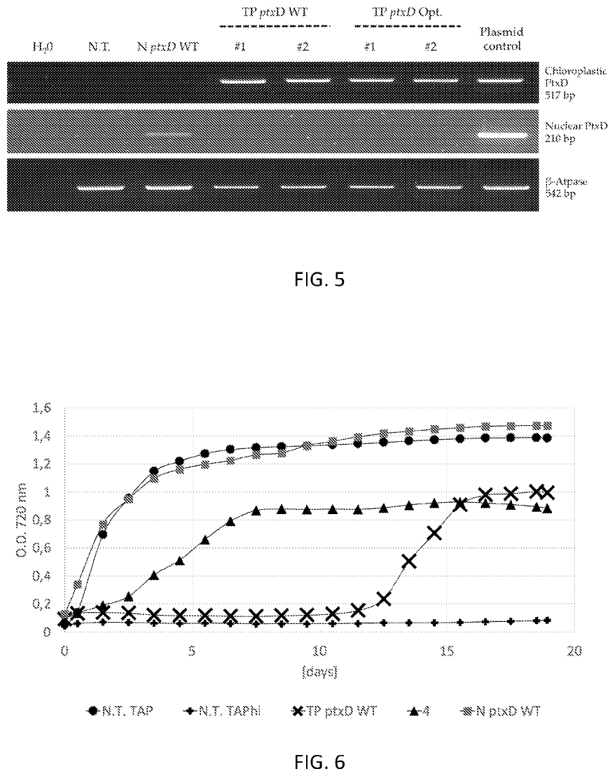

FIG. 5 shows PCR-based genetic characterization of selected transformant lines. Three independent reactions were set up to reveal the presence of the ptxD transgene sequence(s). The upper panel refers to the amplification of the chloroplast codon usage-optimized ptxD sequence while the middle panel refers to the amplification of the nucleus codon usage-optimized ptxD version. A control reaction (lower panel) was set-up to verify the quality of extracted genomic DNA targeting the ATP-ase β subunit gene. Additional control reactions were performed with appropriate primer pairs on vectors containing the ptxD sequences introduced in the algae (IR-int and pChlamy-4 for the chloroplast and nuclear version respectively).

FIG. 6 shows growth curves of transgenic lines expressing the ptxD transgene in different cellular compartments. The selective growth phenotype was assessed by cultivating the algae in TA-Phi medium, following a phosphate-depletion treatment. The untransformed T222 wild type was cultivated in both TA-Phi (cross trace) and TAP (circle trace) media as negative and positive controls, respectively. The nuclear transformant N ptxD WT (square trace) and two independent transplastomic lines carrying the two PTXD versions (TP ptxD WT, x trace and TP ptxD Opt, triangle trace, respectively) were included in the experiment. Traces refer to the average of two technical replicates of growths performed in parallel. Cell density was measured by recording optical density at 720 nm.

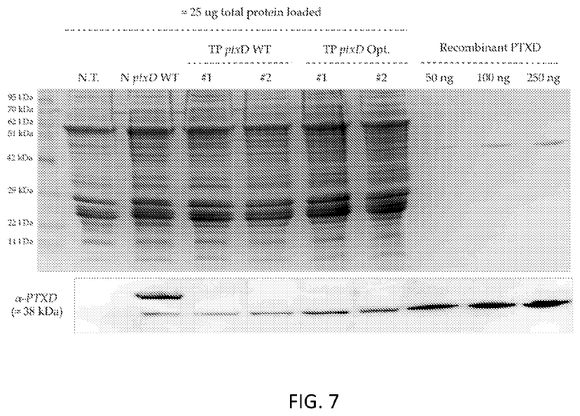

FIG. 7 shows biochemical characterization of produced transgenic lines displaying phosphite-metabolizing activity. A Western blot analysis using the anti-PTXD antibody showed comparable amounts of recombinant enzyme expressed in the transplastomic lines carrying the two PTXD versions. The nuclear transformant N ptxD WT, the untransformed T222 wild type and variable amounts of the recombinant protein expressed in E. coli were included as controls. The upper panel displays a Coomassie-stained SDS-PAGE gel confirming equal amounts of total proteins for all lines (25 μg). Densitometric analysis yielded a relative intensity of the 29 kDa band of 1:1.8:1.7:1.1:2.1:1.9 for, respectively, lanes N.T, NptxD WT, #1, #2, #1, #2 (from left to right).

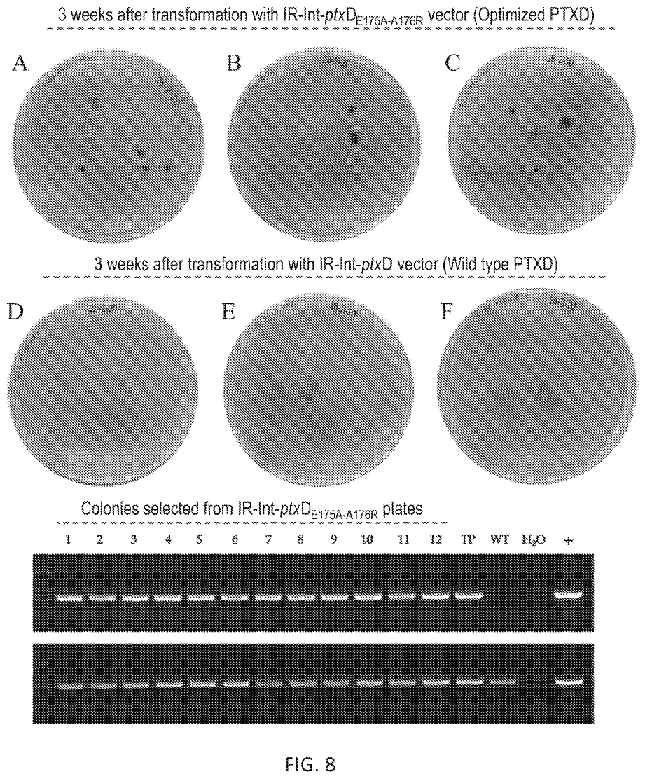

FIG. 8 shows PTXD-based genetic transformation of the chloroplast genome coupled to phosphite metabolism-based selection. One-week phosphorous-starved T222 wild type cells were transformed with the IR-int vectors carrying the two PTXD versions. After 3 weeks, a number of colonies were clearly distinguishable on independent transformation plates for the Int-ptxD_{E175A-A176R} vector (panels A, B and C, circled). No colonies could be retrieved following transformation with the wild type enzyme version (panels D, E and F). True transformation events were confirmed for all selected colonies via PCR-based genetic analysis on genomic DNA targeting the PTXD transgene (1-12, panel G). A positive reaction against the plastid ATP-ase β subunit gene was included (panel H). TP refers to a characterized transplastomic PTXD lines produced in this work; WT is the untransformed T222 wild type control.

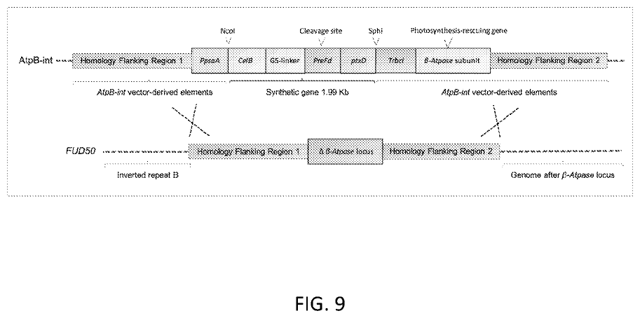

FIG. 9 shows the AtpB-int-CelB-GS-PreFd-ptxD vector containing the in vivo cleavable chimera. The AtpB-int vector backbone contains two homology flanking regions required for the homologous recombination-based transgene insertion (crossed dashed lines) at the level of the disrupted β-Atpase locus (box) in the FUD50 background. This vector carries a functional copy of the β-Atpase gene, required for the restoration of photosynthesis and representing the basis for the selection strategy. The chimeric cassette is composed of the two enzymes (CelB and PTXD) connected by a flexible short amino acid linker (GS), the cis-acting transcriptional regulatory elements (promoter and 5′UTR of psaA, terminator and 3′UTR of rbcL) and the transit peptide of the nuclear encoded preferredoxin protein (PreFd). This latter is the substrate of a plastid-localized protease which produces the scission between the two proteins.

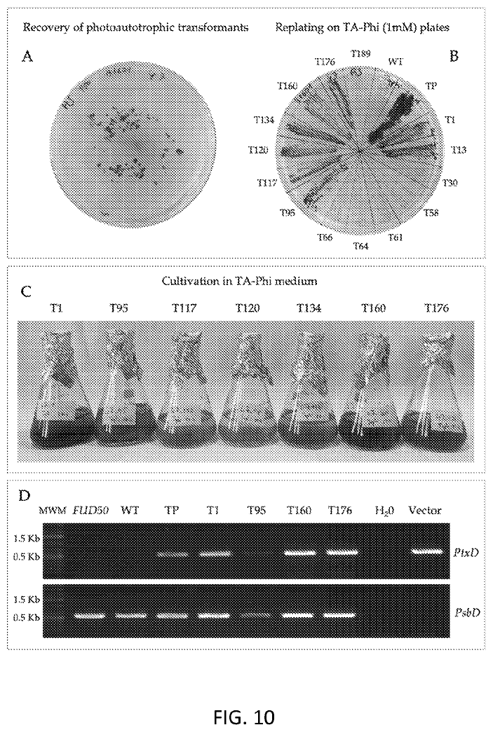

FIG. 10 shows the transformation and selection of photoautotrophic complemented FUD50 transformants. The retrieved photoautotrophic FUD50 transformants (panel A) were transferred to TA-Phi plates (panel B) to enrich the transgenic plastome pool and to screen the Phi-metabolizing activity. Growth analysis in liquid TA-Phi medium (panel C) identified four transformant lines displaying a superior selective growth phenotype (T1, T95, T160 and T176). A control samples corresponding to the previously described PTXD-expressing transplastomic line (TP) was included. A PCR-based analysis revealed the presence of transgenic cassette-associated sequences (517 bp amplicon of the PtxD gene). A control reaction targeting the plastid PsbD locus (554 bp) was included. Control samples included the untransformed FUD50, a WT line and the AtpB-int vector.

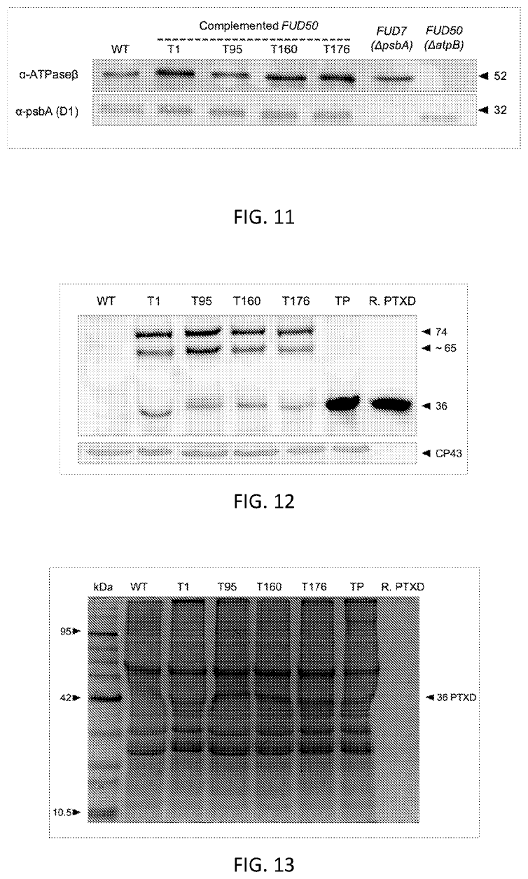

FIG. 11 shows biochemical characterization of complemented FUD50 lines. An immunoblot test was carried out on cell extracts from selected complemented FUD50 transformants (T1, T95, T160, T176) to confirm the synthesis of the β-Atpase subunit responsible for the recovered photoautotrophic competence. A control reaction using an α-PsaA antibody showed comparable amounts of the PSII core subunit D1 protein in the complemented and WT strains. Control samples included protein extracts from WT, FUD7 (a C. reinhardtii mutant devoid of the D1 subunit) and the untransformed FUD50 cells.

FIG. 12 shows immunoblot analysis of the in vivo processing of the chimera. Approximately 25 μg of SDS-PAGE-separated algal proteins were blotted and probed with an anti-PTXD antibody. In all tested lines 3 bands were observed: an upper band of 74 kDa corresponding to the unprocessed chimera, a lower band of 36 kDa corresponding to the released PTXD and an unexpected intermediate size band of approximately 65 kDa, likely originating from an ectopic cleavage of the chimera. Control samples included a previously described PTXD-expressing transplastomic line (TP) and the recombinant PTXD enzyme (100 ng) purified from E. coli. A control reaction with an anti-CP43 antibody shows equal sample loading.

FIG. 13 shows the corresponding Coomassie-stained SDS-PAGE gel of algal total protein extracts corresponding to the loading scheme depicted in the Western blot shown in FIG. 12.

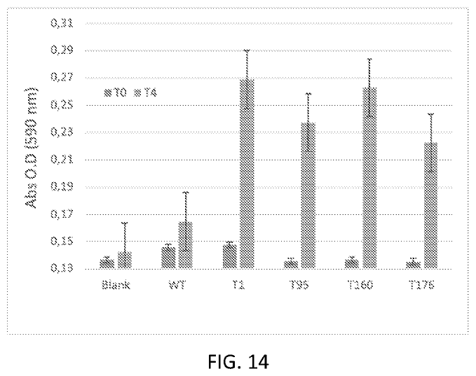

FIG. 14 depicts ex vivo colorimetric cellulose degradation assay using the derivatized carboxymethyl cellulose substrate Azo-CMC. The hydrolytic activity of total algal protein extract was tested using the artificial cellulose substrate Azo-CMC derivatized with the dye Remazol Brilliant Blue. Measurements were taken at time-points zero (T0, blue bars) and after four hours of incubation at 95° C. (T4, orange bars). Values refer to the average of three technical replicates. Absorbance values were recorded at 590 nm. A blank reaction, not including protein extracts, and a negative control, corresponding to algal extracts of wild type cells, were included.

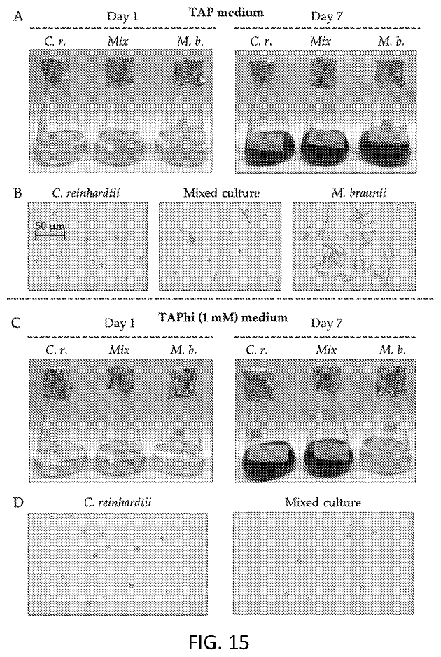

FIG. 15 shows competitive growth in selective TA-Phi medium. The selective growth phenotype conferred by the PTXD enzyme was assessed by inoculating a transplastomic C. reinhardtii line together with the freshwater eukaryotic microalga Monoraphidium braunii as competing species. Panels A (right picture) shows the occurrence of mixed algal populations after seven days of cultivation in the non-selective medium (TAP), as highlighted by the middle microscopy picture in panel B. Panel C shows the exclusive cultivation of transplastomic C. reinhardtii in TA-Phi medium, as highlighted by the prevented growth of M. braunii monoculture (right picture).

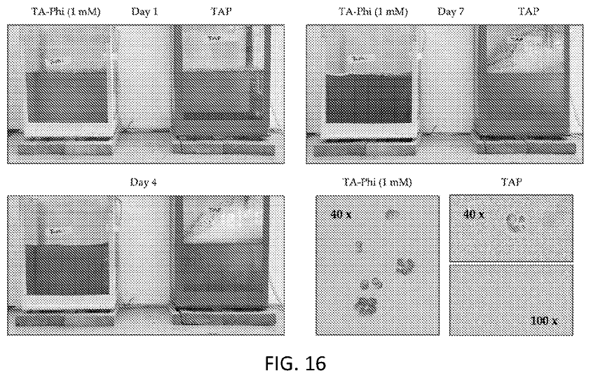

FIG. 16 shows open-air cultivation in non-sterile conditions. The transplastomic line T176 was inoculated in volumes of 4 liters of either TAP (right red tank) or TA-Phi (1 mM, left white tank) media at a cell density of approximately 0.5×10⁶ cells/ml in open tanks kept in non-sterile conditions. Growth in both conditions was followed for 7 days. The TAP culture displayed severe contamination already at day 4, while Phi-fertilization promoted healthy algal growth. Microscopy pictures revealed heavy microbial contamination of the TAP culture (bottom rightmost pictures, 40× and 100× magnification), where sparse algal cells surviving the initial inoculum were surrounded by a dense population of parasites.

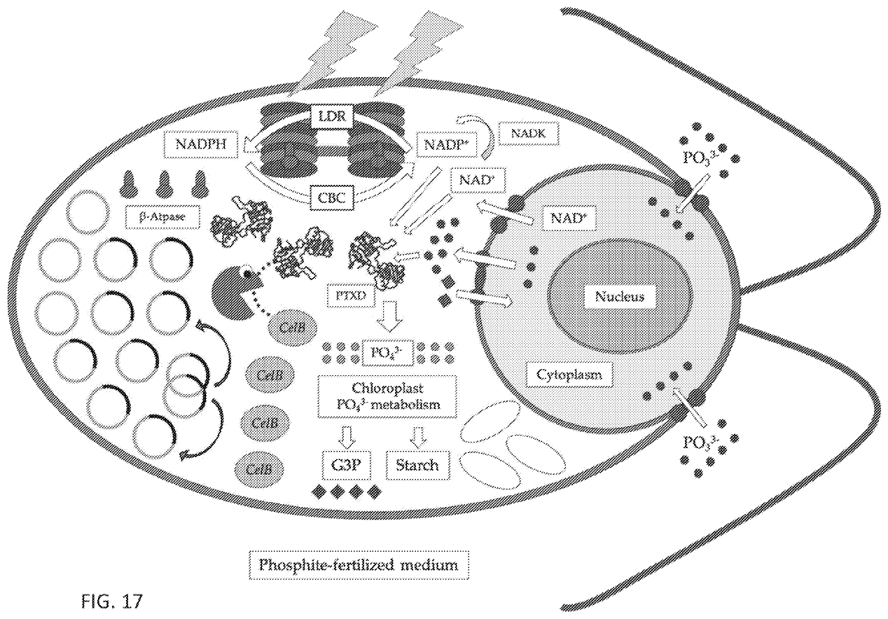

FIG. 17 illustrates a graphical summary of the self-reinforcing genetic system developed in this work. Phosphite ions (PO₃³⁻, circles) entering the algal cytoplasm are imported in the chloroplast in exchange with triose phosphates (G3P, rhombi). The CelB-PTXD chimera is synthetized and processed by a plastid protease causing the release of the two enzymes. The optimized PTXD isoform suited for efficient catalysis in the plastid uses the low abundant NAD+, and NADP+, to convert phosphite in phosphate (PO₄³⁻, dark circles). This latter is used for the synthesis of G3P, starch (white ovals) and nucleic acids. The non-native NADP+ cofactor is produced by the NAD kinase (NADK)-mediated phosphorylation of NAD+ and undergoes a dynamic redox interconversion by the photosynthetic light reactions (LDR) and the Calvin cycle (CBC). The combined selective pressure imposed by the presence of Phi as sole phosphorous source promotes the maintenance of the transformant plastome pool (light grey and black circles, light grey full circles untransformed genome pool), required to sustain the Phi-metabolizing trait conferring the selective growth phenotype (βATPase subunits synthetized following complementation of the non-photosynthetic recipient strain).

The subsequent experimental section describes in detail the optimized PTXD enzyme of the invention and its catalytic efficiency in a NADP⁺-rich environment, such as the chloroplast of Chlamydomonas reinhardtii. The intention is to illustrate certain specific embodiments and application of the optimized PTXD enzyme according to the invention, without limiting the invention.

### EXAMPLE 1: OPTIMIZATION OF THE CATALYTIC ACTIVITY OF PTXD ENZYME IN THE CHLOROPLAST OF CHLAMYDOMONAS REINHARDTII AND EFFECT ON GROWTH PERFORMANCE

### Material and Methods

### Algal Strains and Cultivation Strategies

Chlamydomonas reinhardtii wild type strain T222+ (CC-5101) was obtained from the Chlamydomonas Resource Center and used as recipient strain for all genetic modifications described in this work. Growth experiments were conducted in a laboratory-scale photobioreactor (Multi-Cultivator MC 100-OD, Photon System Instruments, Drasov, Czech Republic) in mixotrophic conditions using an acetate-fertilized Tris-Acetate-Phosphate rich medium (TAP) under the following controlled conditions: continuous light at 200 μmol photons m⁻² s⁻¹ irradiance, 22° C. and constant air bubbling. For general purposes, algae were propagated on TAP-Agar (1.5% w/v) plates containing appropriate antibiotics. Selective growth experiments in phosphite were conducted by substituting the phosphorous source sodium phosphate (K₂PO₄) with sodium phosphite (Na₂HPO₃ 5H₂O. Sigma Aldrich), while maintaining the same ion concentration. Phosphite-fertilized rich and minimal media are hereinafter referred to as TA-Phi and HS-Phi, respectively. A precultivation step in TAP medium preceded the selective growth experiments until a density of 10⁶ cells/ml was reached. Cultures were subsequently pelleted and re-suspended and cultivated in TA medium (phosphorous devoid) for 4 days to exhaust intracellular phosphate stores (phosphate depletion) before being exchanged to TA-Phi medium.

Transformation of the Chloroplast Genome of C. reinhardtii

The ptxD gene sequence, coding for the phosphite dehydrogenase enzyme of Pseudomonas stutzeri WM88, was obtained from the UniProt database (UniProtKB—O69054), optimized according to the AT-rich codon bias of the chloroplast genome of C. reinhardtii [20] using the online tool OPTIMIZER [21] and obtained by GeneScript (Leiden, Netherlands) as a synthetic gene in the pUC57 vector flanked by NcoI and SphI restriction sites.

The nucleotides that were exchanged to create the E175A/A176R double amino acid substitution are illustrated in the following scheme:

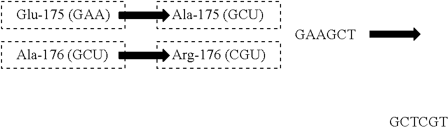

The nucleotide sequence of the chloroplast codon usage-optimized phosphite dehydrogenase (ptxD) gene sequence is the following:

(SEQ ID NO: 1)
ATGTTACCAAAATTAGTTATTACACATCGTGTTCATGATGAAATTTTACA
ATTATTAGCTCCACATTGTGAATTAATGACAAATCAAACAGATTCAACAT
TAACACGTGAAGAAATTTTACGTCGTTGTCGTGATGCTCAAGCTATGATG
GCTTTTATGCCAGATCGTGTTGATGCTGATTTTTTACAAGCTTGTCCAGA
ATTACGTGTTGTTGGTTGTGCTTTAAAAGGTTTTGATAATTTTGATGTTG
ATGCTTGTACAGCTCGTGGTGTTTGGTTAACATTTGTTCCAGATTTATTA
ACAGTTCCAACAGCTGAATTAGCTATTGGTTTAGCTGTTGGTTTAGGTCG
TCATTTACGTGCTGCTGATGCTTTTGTTCGTTCAGGTGAATTTCAAGGTT
GGCAACCACAATTTTATGGTACAGGTTTAGATAATGCTACAGTTGGTATT
TTAGGTATGGGTGCTATTGGTTTAGCTATGGCTGATCGTTTACAAGGTTG
GGGTGCTACATTACAATATCATGAAGCTAAAGCTTTAGATACACAAACAG
AACAACGTTTAGGTTTACGTCAAGTTGCTTGTTCAGAATTATTTGCTTCA
TCAGATTTTATTTTATTAGCTTTACCATTAAATGCTGATACACAACATTT
AGTTAATGCTGAATTATTAGCTTTAGTTCGTCCAGGTGCTTTATTAGTTA
ATCCATGTCGTGGTTCAGTTGTTGATGAAGCTGCTGTTTTAGCTGCTTTA
GAACGTGGTCAATTAGGTGGTTATGCTGCTGATGTTTTTGAAATGGAAGA
TTGGGCTCGTGCTGATCGTCCACGTTTAATTGATCCAGCTTTATTAGCTC
ATCCAAATACATTATTTACACCACATATTGGTTCAGCTGTTCGTGCTGTT
CGTTTAGAAATTGAACGTTGTGCTGCTCAAAATATTATTCAAGTTTTAGC
TGGTGCTCGTCCAATTAATGCTGCTAATCGTTTACCAAAAGCTGAACCAG
CTGCTTGTTAA

The nucleotides that were exchanged via site-directed mutagenesis to create the E175A/A176R double amino acid substitution are underlined.

The mutagenized version of the PTXD enzyme, carrying the substitution of the two adjacent residues Glu-175 and Ala-176 with Ala and Arg, respectively (hereinafter referred to as ptxD_{E175A-A176R}) was produced via site directed mutagenesis of the wild type ptxD sequence contained in the pUC57 vector with a pair of mismatch-containing primers (Ptxd_E175A_A176R FW TTGTTCTGTTTGTGTATCTAAAGCTTTACGAGCATGATATTGTAATGTA GCACCCCAACC (SEQ ID NO:2)); Ptxd_E175A_A176R_RV GGTTGGGGTGCTACATTACAATATCATGCTCGTAAAGCTTTAGATACA CAAACAGAACAA (SEQ ID NO:3)) using the Quick Change Lightning Site-Directed Mutagenesis Kit (Agilent Technologies, Santa Clara, USA) following the manufacturer's instructions. The plasmid used for the chloroplast transformation of the wild type T222 strain is the IR-int vector [19]. This vector contains two homology regions within the inverted repeats of the chloroplast genome of C. reinhardtii, targeting the exon V of the psbA gene (D1 subunit of photosystem II) and the 5S ribosomal RNA and portion of the 23S ribosomal RNA, respectively, enabling a homologous recombination-based transgene insertion at the BamHI site found between these two genomic loci. This vector contains cis-acting regulatory elements derived from endogenous chloroplast genes driving the expression of the transgenes of interest (atpA promoter and 5′ UTR) and to ensure the stability of its transcript (3′ UTR of the RuBisCo large subunit, rbcl), along with the selectable marker gene aadA, providing the detoxifying activity towards the antibiotic spectinomycin. In this work, authors opted for a different cis-acting element pair to drive the expression of the PTXD enzyme(s), derived from the AtpB-int vector that is employed to transform the non-photosynthetic, ATP synthase β subunit-deficient C. reinhardtii strain FUD50 (CC-1185). In particular, the AtpB-int vector provides the highly efficient psaA (subunit A of photosystem I core) promoter. To achieve such cassette configuration for the transgene a two-step subcloning operation was performed starting from the pUC57 vector containing the ptxD sequence, first into the AtpB-int vector and finally into the IR-int vector. To this end, the wild type pUC57-ptxD construct and the herein created mutagenized pUC57-ptxD_{E175A-A176R} version were excised via NcoI and SphI restriction enzymes from the pUC57 vector and firstly ligated into the AtpB-int vector [19] previously digested with the same restriction enzyme pair. By doing so, both PTXD versions were placed under the transcriptional control of the strong, plastid native cis-acting regulatory elements provided by this vector. These larger cassettes, including the PTXD coding sequence(s) and the newly acquired PpsaA and 3′ rebL cis-acting regulatory elements were subsequently excised via ClaI and SmaI restriction sites and ligated into the IR-int vector to produce the IR-Int: ptxD and IR-Int-ptxD_{E175A-A176R}, intended to be used for the transformation of the chloroplast genome of the T222 (CC-5101) wild type C. reinhardtii strain. Transplastomic C. reinhardtii lines were created via the biolistics transformation method using the modified IR-int vectors following an established protocol [22] using plasmid DNA-covered 0.6 μm diameter gold microcarriers (Bio-Rad, Hercules, CA, USA) and a PDS-1000/He gene gun system (Bio-Rad). Transplastomic C. reinhardtii colonies were selected on spectinomycin-containing (100 μg/μl) TAP-Agar plates and individual colonies were subsequently subcultured for several rounds (6-8) on selective plates in order to increase the transgene copy number until full homoplasmy.

Chloroplast transformation of C. reinhardtii exploiting phosphite metabolism-based selection was performed following the same method described above using one-week phosphorous-starved T222 wild type cells. Algae were plated on TA-Phi (1 mM) agar medium and transformed in parallel with the IR-int vectors carrying the two PTXD versions. Genetic characterization of putative transformants was performed via colony PCR directly from three week-old colonies from the original transformation plates using primers specific for the chloroplast codon usage-optimized PTXD coding sequence (described below).

Transformation of the Nuclear Genome of C. reinhardtii

A nuclear transformant, expressing the PTXD enzyme in the cytoplasm, was created via electroporation of T222 C. reinhardtii following a previously described standard transformation protocol and served as additional control in all experiments described in this work. To this end, the pChlamy-4 vector (Life Technologies Corporation, Carlsbad, CA, USA), containing the intron-less ptxD sequence optimized for the codon usage of the nuclear genome of C. reinhardtii under the control of the Hsp70A-RbcS2 chimeric constitutive promoter, was linearized with the ScaI restriction enzyme and 500 ng of plasmid were used to transform C. reinhardtii cells. After a 24 hours recovery in the dark cells were plated on TAP-Agar plates supplemented with the selective agent antibiotic zeocin. Zeocin-resistant clones were subcultured screened for their ability to grow in TA-Phi medium in the presence of phosphite as sole phosphorous source and further genetically and biochemically characterized.

Production of Recombinant PTXD Protein in E. coli

The PTXD enzyme was expressed as a recombinant protein in E. coli to be used as antigen for the production of an anti-PTXD specific polyclonal antibody. To this end, the ptxD gene sequence optimized for nuclear codon usage of C. reinhardtii was amplified via PCR from the pChlamy-4 vector [12] with primers providing restriction sites NdeI and BamHI.

The ptxD cassette flanked by restriction sites was subsequently introduced into the multiple cloning site of the pET28a+ vector (Novagen) via digestion and ligation to express a recombinant PTXD protein carrying an N-terminal polyhistidine (6×) tail. The obtained plasmid pET28a+ptxD-His-tagged was transformed into BL21 (DE3) pLysS E. coli electrocompetent cells and protein overexpression was induced by the addition of isopropyl β-d-1-thiogalactopyranoside (IPTG, 0.1 mM) and culturing of cells for 16 hours at 16° C. Next, the recombinant His-tagged PTXD protein was purified via affinity chromatography on gravity columns packed with a nickel-charged sepharose resin (GE Healthcare, Life Sciences, Pittsburgh, PA, USA). To this end, the crude bacterial lysate was loaded on the column followed by extensive wash in the presence of 20 mM imidazole. The soluble target protein was then eluted with 500 mM Imidazole and 4 elution fractions were pooled, buffer exchanged with PD-10 desalting columns (GE-Healthcare) and concentrated using Amicon Centrifugal filters (Merk Millipore). The recombinantly expressed PTXD protein was checked for purity on Coomassie-stained SDS-PAGE gels and approximately 2 mg of protein were used for rabbit immunization and production of the polyclonal antibody (Davids Biotechnologies, Regensburg, Germany).

### Phenotypical, Genetic and Biochemical Characterization of all Transgenic Lines

All produced transgenic lines underwent an initial phenotypical screening to assessment their ability to selectively grow in a phosphate-devoid, phosphite-fertilized medium. One nuclear transformant and two independent transplastomic lines for each PTXD versions were selected for further characterization. A PCR-based genotyping analysis was performed on genomic DNA extracted with a quick protocol to detect the presence of the ptxD transgene sequence(s). For the nuclear transformant a specific primer pair (Ptxd_596_F CCTCGTCCGACTTCATCCTG (SEQ ID NO:4) and Ptxd_805_R CCCAGTCCTCCATCTCGAAG (SEQ ID NO:5)) was employed to amplify a 210 bp sequence. For the transplastomic lines, a primer pair specific for the chloroplast codon usage-optimized ptxD version was used instead, giving a 517 bp amplicon (chl_PTXD_CDS_FW CCAAAATTAGTTATTACACATCGTG (SEQ ID NO:6) and chl_PTXD_CDS_RV CATGATATTGTAATGTAGCACCC (SEQ ID NO:7). Positive control reactions were conducted with primers specific for the plastid ATP-ase β subunit gene producing a 542 bp amplicon (atpB_CDS_FW GTAAATACTTCAGCTACGAAGAATG (SEQ ID NO:8) and atpB_CDS_RV ATGTTAACAAACAAGACGTATTATTCT (SEQ ID NO:9)).

Selected lines were further characterised biochemically for the expression of the recombinant PTXD protein via western blotting experiments using the custom-made anti-PTXD antibody. To this end, total algal proteins were extracted using an established protocol [24] starting from 15 ml of a C. reinhardtii culture from early exponential phase. Briefly, cells were harvested by centrifugation and the pellet was re-suspended in 300 μl of solution A (0.1% Na₂CO₃) and 200 μl of solution B (5% SDS, 30% sucrose) and 25 ul of β-mercaptoethanol. Samples were incubated at room temperature for 25 min under gentle agitation. Approximately 25 μg of total proteins were separated on a denaturating SDS-PAGE gels followed by Coomassie-staining. In parallel, SDS-PAGE-separated protein samples were transferred to a nitrocellulose membrane using a single buffer device (Bio-Rad) to proceed with the immunodecoration. Blotted membranes were incubated with blocking buffer (PBS 1×, 5% milk powder, 2% TWEEN) and subsequently incubated overnight at 4° C. with the anti-PTXD antibody. Following washes, membranes were incubated with a secondary antibody coupled to a horseradish peroxidase enzyme and signal acquisition was performed with the enhanced chemiluminescence (ECL) method using a ChemiDoc (Bio-Rad) imaging system.

### Bioinformatics Methods

Bioinformatics analyses and protein structure modelling were performed with the PyMOL Molecular Graphics System, Version 2.0 Schrödinger, LLC. (https://www.pymol.org/) using the previously created [25] PDB files of PTXD structures 4E5N, 4E5P and 4E5M, obtained from PDB databank.

### Results

### Site-Directed Mutagenesis of PTXD Cofactor-Binding Amino Acidic Residues

Previous in vitro studies showed that two adjacent residues in the PTXD enzyme, including a negatively charged glutamate, are involved in the selective binding of the NAD⁺ cofactor. The combined substitution of Glutamic Acid 175 and Alanine 176 in PTXD by Alanine and Arginine residues, respectively, resulted in a relaxed cofactor binding selectivity, enabling the concomitant use of NAD⁺ and NADP⁺. From a mechanistic perspective, the introduced double amino acid substitution is assumed to affect the charge distribution around the cofactor-binding pocket, creating an environment that favors the simultaneous docking of both NAD⁺ and NADP⁺ cofactors, while causing only a minor structural alteration of the overall protein topology [25]. The negatively charged phosphate moiety of the NADP⁺ molecule can be more easily accommodated in the pocket as a result of the neutralization of the negative charge of the native residue Glutamate 175 via its substitution with Alanine and the stabilization of the interaction promoted by the introduction of the positively charged residue Arginine in place of Alanine 176 (FIG. 2).

These two amino acid residues have been mutagenized starting from a PTXD template sequence optimized for chloroplast codon usage via a PCR-based, site-directed mutagenesis protocol. By employing a mismatch-containing primer pair, the desired double amino acid substitution in the PTXD coding sequence has been achieved. Both wild type and the mutagenized PTXD versions have been introduced into a wild type C. reinhardtii (T222) strain using the IR-int vector (FIG. 3).

### Effect on Selective Growth of Transplastomic Lines

The assembled IR-int vectors containing the wild type and mutagenized PTXD version carrying the double amino acid substitution (IR-Int-ptxD_{E175A-A176R} construct) were subsequently used to transform the chloroplast genome of T222 C. reinhardtii cells via the biolistic method. Approximately one week after the bombardment, a number of spectinomycin-resistant colonies (>30) appeared on the selective plates containing the algae transformed with both construct versions. At least 10 independent transformants for each construct were selected and further sub-cultivated on TAP-Agar plates in the presence of spectinomycin to increase the transgene copies until near-homoplasmy (6 rounds). Transplastomic lines carrying the mutagenized PTXD version hereafter will be referred to as TP ptxD Opt. A transgenic line containing a nuclear insertion of a transgene encoding the WT PTXD enzyme (hereafter referred to as N ptxD WT) was used as reference strain for the selective growth experiment performed in this work.

Two transplastomic lines and the nuclear transformant line were further analyzed for the ability to selectively grow in liquid medium supplemented with phosphite as sole phosphorous source. Transplastomic lines carrying the mutagenized PTXD version grew in TA-Phi medium at a similar rate as the nuclear counterpart. In contrast, the transplastomic lines expressing the wild type PTXD version (hereafter referred to as TP ptxD WT) grew at a far slower rate in the selective medium. The ability to metabolize phosphite and to convert it into the assimilable form phosphate, is strictly dependent on the expression of the PTXD enzyme, since the untransformed T222 line did not grow either in TA (phosphorous-devoid) or in TA-Phi (phosphite-fertilized) medium (FIG. 4). All lines showing the Phi selective growth phenotype were genetically characterized via PCR and proved positive for the presence of the ptxD transgene sequence (nuclear and chloroplastic) (FIG. 5). To substantiate the observed putative selective growth advantage conferred by mutagenized PTXD version, a growth analysis in a laboratory-scale photobioreactor has been set up. To this end, two independent transplastomic lines for each PTXD version (TP ptxD WT and TP ptxD Opt), the untransformed T222 wild type strain and the nuclear transformant N ptxD WT, were included. All transgenic lines were cultivated in TA-Phi (phosphite-fertilized) medium under continuous illumination with 200 μE light intensity and their growth was monitored. A positive and a negative control, consisting in the untransformed T222 wild type strain cultivated in TAP (phosphate-fertilized) and TA-Phi medium, respectively, were included. With this experimental set-up it is possible to reproduce the previously inferred differences in the selective growth performance between lines. As shown in FIG. 6, the N ptxD WT line grew in TAPhi medium (phosphite-fertilized) at a similar rate as the untransformed T222 wild type strain does in TAP medium (phosphate-fertilized).

This observation indicates that the availability of phosphite is not limiting in the cytoplasmic compartment of the alga and that the amount of nuclear-encoded recombinant PTXD enzyme is sufficient to efficiently catalyze the conversion of phosphite into phosphate by using the available NAD⁺ cytosolic pool. The inability to grow in TA-Phi medium displayed by the untransformed algal strain demonstrates that the PTXD activity is strictly required for the Phi growth phenotype. The TP ptxD Opt transplastomic lines displayed an intermediate growth phenotype but significantly faster than that of the TP ptxD WT transplastomic line expressing the wild type PTXD enzyme. The latter displayed the slowest growth phenotype, reaching the plateau phase about 14 days later than the strain carrying the mutagenized PTXD counterpart, and 16 days after the PTXD WT nuclear transformant.

### Faster Selective Growth is Related to a Higher Catalytic Efficiency of the Mutagenized PTXD Version

All transgenic lines have been characterized via Western blotting using the anti-PTXD antibody to assess the levels of recombinant enzyme. For this determination, equal amounts of total algal protein extracts based on Bradford's assay have been loaded. Loading of each lane base of densitometry of the Coomassie-stained gel (FIG. 7) has been further normalized. Upon normalization, no striking differences in the levels of PTXD protein among the lines have been observed, suggesting that the growth advantage displayed by the TP ptxD Opt lines respect to TP ptxD WT transplastomic lines is derived from a superior catalytic efficiency of this modified enzyme version rather than from a higher level of PTXD in the plastid. An unexpected higher molecular weight band was observed in the N ptxD WT nuclear transformant, which could correspond to a highly glycosylated form of the enzyme.

### The Optimized PTXD Version Enables Fast and Reliable Recovery of Transformants

Next it has been investigated whether the modified PTXD enzyme version could serve as a reliable selectable marker for the genetic transformation of the algal plastome using a phosphite metabolism-based selection strategy. To this end, in parallel, one-week phosphorous-starved cells in with the IR-vectors carrying the wild type and modified PTXD version on TA-Phi (1 mM) agar plates has been transformed, as previously described [14]. As shown in FIG. 8, the transformation with the Int-ptxD_{E175A-A176R} vector resulted in the formation of colonies at 3 weeks post-bombardment (panels A, B and C), while no colonies appeared following transformation of the wild type PTXD version (panels D, E and F). In total, three independent transformations were performed for each vector version and, in the case of IR-Int-ptxD_{E175A-A176R}, 12 larger colonies were assessed via genetic analysis. The PCR analysis confirmed the presence of the PTXD transgene in all of them, confirming their identity as true transformation events (FIG. 8, panel G). These results clearly support the value of the modified PTXD as a superior selectable marker gene compared to the native enzyme.

The optimized enzyme version enables faster selective growth of transplastomic algae in nutrient (phosphate)-limited conditions, thus allowing this system to be implemented in large scale cultivation. In addition, the modified PTXD transgene favors a faster and reliable recovery of transplastomic transformants, fully establishing this system an alternative, environmentally friendly selection method that will replace antibiotic resistance gene-based protocols.

### EXAMPLE 2: STABLE EXPRESSION OF A CLEAVABLE HYPERTHERMOPHILIC ENDOGLUCANASE-PTXD CHIMERA IN THE CHLOROPLAST OF CHLAMYDOMONAS REINHARDTII

An exemplary cleavable hyperthermophilic endoglucanase-PTXD chimera and its stable expression in the chloroplast of Chlamydomonas reinhardtii is here reported. Endoglucanase CelB [29] from the extremophile bacterium Thermotoga neapolitana has been expressed in the chimera together with the modified ptxD gene.

Hyperthermophilic hydrolases find industrial application in the decomposition of β-1,4 glucose polymers of lignocellulose, where endoglucanases play a fundamental role by attacking the crystalline cellulose fibers, releasing smaller oligomers that are further processed by auxiliary hydrolytic enzymes. The extreme optimal physiological working range of CelB (>95° C.) keeps the enzyme inactive within the mesophilic algal host, while its sequestration in the chloroplast prevents detrimental effects towards cellular components susceptible to hydrolysis.

As a consequence, the enzyme activity is manifested once the protein is released from the dried algal biomass and it encounters suitable chemo-physical conditions reproduced in the industrial processes. Lignocellulose is a structurally heterogeneous material made of interconnected cell wall components which confer a high degree of compactness. For this reason, thermal pre-treatments of the raw biomass are usually employed to promote the unwinding of its compact fibers and to grant easier access to hydrolytic enzymes towards their native substrates.

### Materials and Methods

### Algal Strains, Cultivation Strategies and Transformation of the Chloroplast Genome

The non-photosynthetic, acetate-requiring C. reinhardtii FUD50 mutant (CC-1185, mating type +) was used in this work as recipient strain for all genetic manipulations (Chlamydomonas Resource Center, University of Minnesota, St. Paul, MN, USA). This homoplasmic, non-spontaneously revertant mutant, carries a deletion affecting the single copy plastid β-Atpase subunit gene located at the upper margin of the inverted repeat B [26], which prevents the assembly of a functional ATPase complex. This photosensitive mutant cannot grow photoautotrophically and must be propagated heterotrophically in the dark on rich Tris-Acetate Phosphate (TAP) medium [27]. Chloroplast transformation of this mutant relies on a cognate vector containing a functional copy of β-Atpase subunit gene as selectable marker and exploits a selection based on the restoration of photoautotrophy. The previously described transplastomic line expressing the optimized PTXD isoform (CC-5101 background, mating type +, here referred to as TP) was included as control in immunological experiments. Different cultivation media were used depending on the selective pressure required at each experimental step. Transformation of the FUD50 plastome was performed via biolistics, using the Biolistic® PDS-1000/He Particle Delivery System (BioRad, Hercules, CA, USA), to deliver foreign DNA precipitated on gold microcarriers following an established protocol [22]. In total, 5 μg of purified plasmid DNA were used to transform 2.5 1×10⁷ heterotrophically-grown C. reinhardtii cells. Photosynthesis-rescued colonies were selected on high salt (HS) minimal medium, under light regime of 80 μmol photons m⁻² s⁻¹ at 23° C., under 16/8 h photoperiod. Transformants were subsequently screened for their Phi-metabolizing ability in mixotrophy on a modified TA-agar medium containing Phi as sole phosphorous source (hereafter referred to as TA-Phi, 1 mM, Na₂HPO₃·5H₂O, Sigma Aldrich).

### In Silico Design of the Chimeric Protein Fusion and Transformation Vector System

The simultaneous insertion of the CelB and ptxD transgenes in the plastome was achieved by designing a chimeric cassette consisting of the two proteins fused by a short flexible polypeptide linker (FIG. 9) and a cleavable substrate recognized by a plastid protease. The synthetic transgenic cassette was assembled in silico using the amino acid sequence of the following elements (from N- to C-terminus): the mature endoglucanase CelB from Thermotoga neapolitana lacking the first 17 residues (secretory signal peptide) (254 AA, Uniprot P96492) [28, 29] the GS peptide linker (14 AA, GGGGSSGGGGGGSS, SEQ ID NO:10) [30], the transit peptide sequence of the nuclear-encoded preferredoxin precursor protein (AA 1-32, PreFd, Uniprot A8IV40, CHLRE_14g626700v5) and the PTXD enzyme isoform (336 AA, Uniprot 069054) from Pseudomonas stutzeri WM88 [10] carrying two amino acid substitutions within the cofactor-binding pocket [16]. The obtained polynucleotide (hereafter referred to as CelB-GS-PreFd-ptxD) was reverse-translated into a codon-optimized sequence for expression from the AT-rich C. reinhardtii plastome using the online tool OPTIMIZER [21] using the codon usage table from the Kazusa database [31]. The retrieved nucleotide sequence was flanked by NcoI and SphI restriction sites at its 5′ and 3′ ends, respectively, to facilitate subsequent cloning operations (supplemental materials). Occasionally introduced NcoI and SphI sites were manually replaced by synonymous codons. The synthetic gene cassette was produced by GeneScript (Leiden, The Netherlands) and provided in the pUC57 vector. The CelB-GS-PreFd-ptxD cassette was excised and ligated in the AtpB-int vector to produce the AtpB-int-CelB-GS-PreFd-ptxD construct. In this way, the CelB-GS-PreFd-ptxD cassette was introduced between the two homology flanking regions required for the homologous recombination-based transgene insertion and acquired the following strong, plastid native cis-acting transcriptional regulatory elements: the PpsaA promoter and 5′UTR of psaA (subunit A of photosystem I) and the TrbcL terminator and 3′UTR of rbcL (RuBisCo large subunit) [32,19].

The nucleotide sequence of the chloroplast codon usage-optimized CelB-GS-PreFd-ptxD chimeric cassette introduced in the ATP-int vector and used to transform the FUD50 plastome. NcoI and SphI restriction sites are found at the 3′ and 5′ ends (underlined), respectively. Elements are placed in the following order: the CelB enzyme, GS linker, PreFd element corresponding to the transit peptide of the preferredoxin precursor, the 3× Flag tag and the optimized PTXD enzyme, carrying the two amino acid substitutions involved in cofactor binding (E175A and A176R) are part of a single open reading frame leading to the synthesis of chimeric polypeptide.

(SEQ ID NO: 11)
CCATGGCTGAAGTTGTTTTAACAGATATTGGTGCTACAGATATTACATT
TAAAGGTTTTCCAGTTACAATGGAATTAAATTTTTGGAATGTTAAATCA
TATGAAGGTGAAACATGGTTAAAATTTGATGGTGAAAAAGTTCAATTT
TATGCTGATATTTATAATATTGTTTTACAAAATCCAGATTCATGGGTTC
ATGGTTATCCAGAAATTTATTATGGTTATAAACCTTGGGCTGCTCATAA
TTCAGGTACAGAAATTTTACCAGTTAAAGTTAAAGATTTACCAGATTTT
TATGTTACATTAGATTATTCAATTTGGTATGAAAATGATTTACCAATTA
ATTTAGCTATGGAAACATGGATTACACGTAAACCAGATCAAACATCAG
TTTCATCAGGTGATGTTGAAATTATGGTTTGGTTTTATAATAATATTTT
AATGCCAGGTGGTCAAAAAGTTGATGAATTTACAACAACAATTGAAAT
TAATGGTTCACCAGTTGAAACAAAATGGGATGTTTATTTTGCTCCTTGG
GGTTGGGATTATTTAGCTTTTCGTTTAACAACACCAATGAAAGATGGT
CGTGTTAAATTTAATGTTAAAGATTTTGTTGAAAAAGCTGCTGAAGTT
ATTAAAAAACATTCAACACGTGTTGAAAATTTTGATGAAATGTATTTTT
GTGTTTGGGAAATTGGTACAGAATTTGGTGATCCAAATACAACAGCTG
CTAAATTTGGTTGGACATTTAAAGATTTTTCAGTTGAAATTGGTGAAG
GTGGTGGTGGTTCATCAGGTGGTGGTGGTGGTGGTTCATCAATGG
CTATGGCTATGCGTTCAACATTTGCTGCTCGTGTTGGTGCTAAACCAGC
TGTTCGTGGTGCTCGTCCAGCTTCACGTATGTCATGTATGGCTATGGAT
TATAAAGATCATGATGATTATAAAGATCATGATGATTATAAAGATG
ATGATGATAAATTACCAAAATTAGTTATTACACATCGTGTTCATGATG
AAATTTTACAATTATTAGCTCCACATTGTGAATTAATGACAAATCAAA
CAGATTCAACATTAACACGTGAAGAAATTTTACGTCGTTGTCGTGATG
CTCAAGCTATGATGGCTTTTATGCCAGATCGTGTTGATGCTGATTTTTT
ACAAGCTTGTCCAGAATTACGTGTTGTTGGTTGTGCTTTAAAAGGTTTT
GATAATTTTGATGTTGATGCTTGTACAGCTCGTGGTGTTTGGTTAACAT
TTGTTCCAGATTTATTAACAGTTCCAACAGCTGAATTAGCTATTGGTTT
AGCTGTTGGTTTAGGTCGTCATTTACGTGCTGCTGATGCTTTTGTTCGT
TCAGGTGAATTTCAAGGTTGGCAACCACAATTTTATGGTACAGGTTTA
GATAATGCTACAGTTGGTATTTTAGGTATGGGTGCTATTGGTTTAGCTA
TGGCTGATCGTTTACAAGGTTGGGGTGCTACATTACAATATCATGCTC
GTAAAGCTTTAGATACACAAACAGAACAACGTTTAGGTTTACGTCAAG
TTGCTTGTTCAGAATTATTTGCTTCATCAGATTTTATTTTATTAGCTTTA
CCATTAAATGCTGATACACAACATTTAGTTAATGCTGAATTATTAGCTT
TAGTTCGTCCAGGTGCTTTATTAGTTAATCCATGTCGTGGTTCAGTTGT
TGATGAAGCTGCTGTTTTAGCTGCTTTAGAACGTGGTCAATTAGGTGGT
TATGCTGCTGATGTTTTTGAAATGGAAGATTGGGCTCGTGCTGATCGTC
CACGTTTAATTGATCCAGCTTTATTAGCTCATCCAAATACATTATTTAC
ACCACATATTGGTTCAGCTGTTCGTGCTGTTCGTTTAGAAATTGAACGT
TGTGCTGCTCAAAATATTATTCAAGTTTTAGCTGGTGCTCGTCCAATTA
ATGCTGCTAATCGTTTACCAAAAGCTGAACCAGCTGCTTGTTAAGCAT
GC

### Genetic and Biochemical Characterization of Transformants

PCR-based analyses of transformant lines were performed on genomic DNA extracted following a quick protocol [23]. The following oligonucleotides for the ptxD (517 bp amplicon chl_PTXD_CDS_FW CCAAAATTAGTTATTAC ACACATCGTG (SEQ ID NO:6) and chl_PTXD_CDS_RV CATGATATTGTAATGTAGCACCC (SEQ ID NO:7), CelB (536 bp amplicon, CelB_777_CDS_FW GCTGAAGTTGTITTAACAGATATTG (SEQ ID NO:12 and CelB_777_CDS_RV CAACCCCAAGGAGCAAAATAAAC (SEQ ID NO: 13) and plastid control gene PsbD (554 bp amplicon, psbD_CDS_FW GAGCGTTACCACGTGGTAATA (SEQ ID NO:14) and psbD_CDS_RV GTTCTTTGCACCTAGTTTCGG (SEQ ID NO:15) were used. Total algal proteins were extracted following an established protocol [24]. For immunoblot experiments, approximately 25 μg of proteins were separated via SDS-PAGE and blotted on nitrocellulose membranes. The α-β-Atpase and α-PsbA (D1) (Agrisera, Vännäs, Sweden) antisera, along with the previously described α-PTXD [18] and α-PsbC (CP43, integral thylakoid membrane chlorophyll binding protein) were used as primary antibodies. Blots were developed using a secondary alkaline phosphatase-conjugated «-rabbit antibody, (Sigma Aldrich) or a horseradish peroxidase-conjugated antibody (Agrisera) via the ECL method.

### Ex Vivo Colorimetric Cellulose Degradation Assay

The activity of the endoglucanase was assessed ex-vivo using a standard colorimetric assay by quantifying the amount of reduced sugar equivalents based on p-hydroxybenzoic acid hydrazide (PAHBAH test, Sigma Aldrich) [28]. Total soluble proteins were extracted from 0.6 mg of dried algal powder in 1 ml of non-denaturing phosphate-citrate buffer (100 mM, pH 6.3) supplemented with 0.5% TWEEN and the protease inhibitor phenylmethylsulfonyl fluoride (PMSF, 100 mM) and followed by sonication. Cell debris were pelleted at high speed and 10 μl of the supernatant were added to 90 μl of phosphate-citrate buffer containing 1% of carboxymethyl cellulose (Sigma Aldrich) and incubated at 100° C. for 4 hours [29].

Assays were stopped by adding 9 volumes of a 0.01% w/v PAHBAH NaOH solution and incubation at 100° C. for 10 minutes. 200 μl were aliquoted in a 96-well microtiter plate and absorbance was recorded in a plate reader spectrophotometer (Infinite M Plex, TECAN, Männendorf, Switzerland) at 410 nm. Enzyme activity was expressed as enzymatic units (μmol of reducing sugar equivalents released per minute) normalized to the microalgal dry biomass weight. A standard calibration curve was prepared using defined amounts of glucose (0, 5, 10, 20, 40, 80 μg/ml). A parallel ex vivo hydrolysis assay using the artificial carboxymethyl cellulose substrate derivatized with the Remazol Brilliant Blue dye (Azo-CMC Megazyme, Bray, Ireland) was run at 95° C. using same amounts of total algal protein extracts and reaction parameters. Reactions were stopped by the addition of 2.5 volumes of a precipitation solution prepared according to manufacturer's manual, followed by centrifugation at 16.000 g for 10 minutes. 200 μl of the supernatants were aliquoted in a 96-well microtiter plate and the absorbance recorded at 590 nm. Control reactions included a blank sample devoid of cell extracts and a wild type C. reinhardtii extracts, as this species naturally secretes cellulases.

### Competitive Growth Experiments and Open-Air Cultivation

Experiments were performed in mixotrophic cultivation using the transplastomic C. reinhardtii line T176 and the freshwater green microalga Monoraphidium braunii (EPSAG Collection of Algae, University of Göttingen, Germany, SAG strain number 2020-7d) as a natural contaminating species. Non-phosphorous starved 1×10⁷ cells of M. braunii and C. reinhardtii cells were inoculated in 150 ml of TAP and TA-Phi (1 mM) sterile liquid media as monocultures and mixed in a 1:1 ratio and cultivated for 7 days. Open-air cultivation of line T176 was performed in a greenhouse environment in non-sterile, non-controlled temperature and light conditions by inoculating 4 liters volume of either TAP or TA-Phi (1 mM) media with 0.5×10⁶ cells/ml with stirring and air supplied through a gas sparger.

### Statistical Analysis

Significance analysis was performed using either Student's t test or ANOVA followed by Tukey's post-test, in GraphPad Prism software. Error bars represent the standard deviation.

### Results

### The Photoautotrophic Transformants Thrive in TA-Phi Medium

Following transformation of the heterotrophically-grown FUD50 cells with the AtpB-int-CelB-GS-PreFd-ptxD vector (described in FIG. 9) and selection on minimal medium (HS) plates over 200 photoautotrophic colonies (FIG. 10, panel A) were recovered. The Phi-metabolizing ability of transformants was initially assessed on TA-Phi-Agar plates, followed by 3 replating rounds to enrich the transgenic plastome pool.

Out of fourteen selected transformant lines, seven displayed fast growth on TA-Phi plate (T1, T95, T117, T120, T134, T160, T176) (FIG. 10, panel B). Growth analysis in selective TA-Phi liquid medium identified four lines (T1, T95, T160, 176) displaying faster growth (FIG. 10, panel C). These were used in the following characterization steps. A PCR-based analysis revealed the presence of transgene-associated elements (FIG. 10 D), while an immunoblot test confirmed the rescued synthesis of the β-Atpase subunit in the complemented FUD50 transformants (FIG. 11).

### Proteolytic Processing In Vivo

The occurrence of the in vivo proteolytic processing of the chimera was investigated via immunodecoration analysis of total algal protein extracts with an anti-PTXD antibody. Three reactive bands in all transformants were detected (FIG. 12). While the upper (74 kDa) and lower (36 kDa) bands correspond to the unprocessed chimera and the released PTXD, respectively, the identity of the intermediate band of approximately 65 kDa is unknown. This latter likely originates from the activity of a plastid protease towards a cleavage site different from the pre-ferredoxin maturation site in the linker peptide and localized within the CelB sequence, as low molecular weight PTXD degradation products in immunoblots were never observed. FIG. 13 shows the corresponding Coomassie-stained SDS-PAGE gel of algal total protein extracts corresponding to the loading scheme depicted in the Western blot shown in FIG. 12.

### Algal Protein Extracts Display Cellulolytic Activity

To quantify the hydrolytic activity of the CelB endoglucanase expressed in the transformant chloroplast, the amount of released reducing sugars was evaluated using the PAHBAH test (see material and methods for details) by incubating crude cell protein extracts with 1% w/v carboxymethyl cellulose for 4 h at 100° C. As shown in table I, the CelB specific activity was very similar in 3 (T1, T95 and T160) over 4 independent transformant lines (6.9-8.7 U g⁻¹ DW), while it was slightly lower in line T176.

Consistent results were obtained by testing the same total protein extracts in a parallel ex vivo colorimetric assay using the Remazol Brilliant Blue-derivatized artificial carboxymethyl cellulose (Azo-CMC). The β-1,4-endoglucanase-catalyzed de-polymerization of the Azo-CMC substrate could be detected in all transformant lines after 4 hours of sample incubation at 100° C. (FIG. 14).

These values are analogous to those obtained in a recent work in which the same endoglucanase was expressed in the algal chloroplast using an antibiotic resistance-based transformation protocol [28], thus highlighting comparable levels of recombinant enzyme accumulation and catalytic activity in the system object of the present invention.

Table 1 reports an estimation of enzyme yield of selected CelB-expressing transplastomic C. reinhardtii lines. Proteins extracts obtained from dry biomass were incubated with a 1% solution of carboxymethyl cellulose (CMC) to determine the hydrolytic activity of the endoglucanase enzyme. Enzyme yield is expressed as enzyme units (U, μmol of reducing sugar equivalents released per minute upon hydrolysis) per grams of dry weight (DW) of microalga. A glucose calibration curve (5-80 μg ml⁻¹) was used to determine the amount of released reducing sugars (μg ml⁻¹ of glucose-equivalent). Data are expressed as mean±SD, n=3; values marked with the same letters are not significantly different from each other (ANOVA test, P<0.05).

TABLE 1

| Line | Enzyme yield (U g⁻¹ DW) |
|---|---|
| T1 | 7.37 ± 0.51^{a} |
| T95 | 6.86 ± 0.33^{a} |
| T160 | 8.57 ± 0.34^{b} |
| T176 | 3.74 ± 0.41^{c} |

Fertilization with Phi Prevents Contamination by a Competing Species and Enables Open-Air Cultivation

The results of the competitive growth between the CelB-PTXD chimera-expressing C. reinhardtii transformant and the wild type microalga Monoraphidium braunii, are shown in FIG. 15. While cultivation in phosphate-containing (TAP) medium for 7 days promoted equal viability of the two species (panel C, left picture) resulting in comparable final cellular densities (plateau stage≈1.5×10⁷ cells/ml, detail shown in microscopy pictures in panel B), fertilization with phosphite (TA-Phi medium) prevented the growth of the competing species (panel C, left picture). Cultivation in TA-Phi medium favored growth of C. reinhardtii only, while the M. braunii monoculture showed only a residual cell density at day 7 (panel C, left picture), consistent with a stunted growth due to exhaustion of internal phosphate stores. As expected, the mixed inoculum resulted in the exclusive growth of transplatomic ptxD-expressing C. reinhardtii cells (panel D, left picture). This result implies that the phosphate produced from the phosphite provided in the medium and converted in the chloroplast of transplastomic C. reinhardtii did not become available to other organisms in the same culture. This ensured the selective and exclusive growth of the ptxD-expressing species.

Large-scale algal cultivation for industrial purposes is usually carried out in closed photobioreactors, which require careful monitoring and costly sterilization procedures, particularly when mixotrophic conditions are employed to achieve maximal biomass productivity. Alternatively, a low-cost, high value solution consists in outdoor cultivation in open ponds, where strict autotrophic growth of dominant target algal species is mandatory in order to avoid contamination by fungi and/or bacteria resulting into culture collapse.

Here the issues of axenic and non-sterile algal cultivation via a heterologous expression system where the chimeric moiety of PTXD supported growth in media containing phosphite as the sole phosphorous source, preventing contamination by other phosphate-requiring, non ptxD-expressing organisms, were addressed. In order to verify the robustness of the axenic growth system in supporting the accumulation of algal biomass containing a recombinant cellulase, a mixotrophically C. reinhardtii line expressing the chimeric construct has been grown in open tanks using untreated, unfiltered, non-sterile tap water supplemented with salts, acetate as a reduced carbon source and phosphite (1 mM).

Tanks were placed in the greenhouse where several plant species are found and microbial contaminants from soil are widespread.

FIG. 16 shows the result of the greenhouse experiment: cultivation in TA-Phi medium yielded a pure green C. reinhardtii culture after 7 days, while growth in TAP resulted, already at day 4, in a brownish and foamy culture which completely collapsed at day 7. Microscopic inspection revealed a dense population of bacterial rods with only sparse green algal cells survivors in the latter, while the TA-Phi culture contained transgenic Chlamydomonas cells only (Figure C, bottom right and left, respectively).

This result supports the effectiveness of the PTXD element introduced in the algal plastome as part of an in vivo cleavable chimera as an efficient tool to promote selective growth of the target species.

The above results strongly demonstrate that a novel system for expressing a high-value biocatalyst in the algal chloroplast by introducing multiple exogenous DNA sequences via a single transformation event, ensuring genetic stability through a sustainable selective pressure has been obtained. In the chimeric construct of the invention the enzyme activity conferred by the ptxD sequence plays a strategic dual function of genetic stabilizer and growth selector. Since the chimeric ORF is placed under transcriptional control of a single pair of cis-acting regulatory elements, the synthesis of the two enzymes forming the cleavable fusion protein is required to sustain the exclusive algal growth via the assimilatory oxidation of phosphite exerted by the product of the downstream element ptxD. Hence, fertilization with phosphite acts as a low-cost selective agent which forces the maintenance of the bifunctional transgene in the algal plastome (see FIG. 17) ensuring the accumulation of the hydrolytic enzyme.

This proof of concept study offers a new solution for stable chloroplast engineering of the model species C. reinhardtii, enabling multiple desirable traits to be introduced without antibiotic resistance-based transformation and selection protocols. The self-sustaining genetic mechanism developed in this work is suitable for the large-scale production of high-value recombinant products and obviates some of the main issues that currently hinder the development of microalgal biotechnology.

### REFERENCES

[1] Rasala, B. A., & Mayfield, S. P. (2015). Photosynth Res, 123(3), 227-239.
[2] Esland, L., Larrea-Alvarez, M., & Purton, S. (2018). Biology (Basel), 7(4).
[3] Maul, J. E., et al. Plant Cell, 2002. 14(11): p. 2659-79.
[4] Doron, L., N. Segal, and M. Shapira. Front Plant Sci, 2016. 7: p. 505.
[5] Verma, D. and H. Daniell. Plant Physiol, 2007. 145(4): p. 1129-43.
[6] Schroda, M., Cells, 2019. 8(12).
[7] Carney, L. T., & Lane, T. W. (2014). Front Microbiol, 5, 278.
[8] Wang, H., et al. Bioresour Technol, 2013. 128: p. 745-50.
[9] McBride, R. C. (2014). Industrial Biotechnology, v. 10 (n. 3), pp. 221-227.
[10] Costas, A. M., White, A. K., & Metcalf, W. W. (2001). J Biol Chem, 276(20), 17429-17436.
[11] López-Arredondo, D. L., & Herrera-Estrella, L. (2012). Nat Biotechnol, 30(9), 889-893.
[12] Loera-Quezada, M. et al. (2016). Plant Biotechnol J, 14(10), 2066-2076.
[13] Changko, S., et al. Appl Microbiol Biotechnol, 2019.
[14] Sandoval-Vargas, J. M., et al. Algal Research, 2018. 33: p. 291-297.
[15] White, A. K., & Metcalf, W. W. (2007). Annu Rev Microbiol, 61, 379-400.
[16] Woodyer, R., W. A. van der Donk, and H. Zhao. Biochemistry, 2003. 42(40): p. 11604-14.
[17] Johannes, T. W., R. D. Woodyer, and H. Zhao. Appl Environ Microbiol, 2005. 71(10): p. 5728-34.
[18] Cutolo, E., et al., (2020). Plants (Basel), 9(4).
[19] Michelet, L., et al. Plant Biotechnol J, 2011. 9(5): p. 565-74.
[20] Nakamura, Y., T. Gojobori, and T. Ikemura. Nucleic Acids Res, 2000. 28(1): p. 292.
[21] Puigbo, P., et al., Nucleic Acids Res, 2007. 35 (Web Server issue): p. W126-31.
[22] Guzman-Zapata, D., et al. Methods Mol Biol, 2016. 1385: p. 69-85.
[23] Cao, M., et al. Protoplasma, 2009. 235(1-4): p. 107-10.
[24] Bertalan, I., et al. J Biotechnol, 2015. 195: p. 60-6.
[25] Zou, Y., et al., Biochemistry, 2012. 51(21): p. 4263-70.
[26] Woessner, J. P., Gillham, N. W., & Boynton, J. E. (1986). Gene, 44(1), 17-28.
[27] Kropat, J., et al. (2011). Plant J, 66(5), 770-780.
[28] Benedetti, M. et al. (2020). Plant Biotechnol J, p. 1-14.
[29] Bok, J. D., Yernool, D. A., & Eveleigh, D. E. (1998). Appl Environ Microbiol, 64(12), 4774-4781.
[30] Chen, X., Zaro, J. L., & Shen, W. C. (2013). Adv Drug Deliv Rev, 65(10), 1357-1369.
[31] Holcomb, D. D., Alexaki, A., Katneni, U., & Kimchi-Sarfaty, C. (2019). Infect Genet Evol., 73, 266-268.
[32] Barnes, D., Franklin, S., Schultz, J., Henry, R., Brown, E., Coragliotti, A., & Mayfield, S. P. (2005). Mol Genet Genomics, 274(6), 625-636.

1. A ptxD nucleotide sequence encoding for the NAD⁺ dependent-phosphite dehydrogenase D enzyme of Pseudomonas stutzeri WM88 (Gene Bank AF061070.1) comprising:
the substitution of the GAA codone encoding for glutamic acid 175 by a codone encoding for alanine residue;
the substitution of the GCT codone of alanine 176 by a codone encoding for arginine residue;
the optimization of each codon according to the AT-rich codon bias of the chloroplast genome of microalgae;
wherein said nucleotide sequence is a DNA or RNA sequence.

2. The ptxD nucleotide sequence according to claim 1 wherein said microalgae belong to the genus Chlamydomonas or Chlorella, preferably to the species Chlamydomonas reinhardtii, Chlorella vulgaris, Chlorella sorokiniana or Chlorella ohadii.

3. The ptxD nucleotide sequence according to claim 1 comprising SEQ ID NO:1.

4. The plasmid or a vector comprising the ptxD nucleotide sequence according to claim 1.

5. A method of use of the ptxD nucleotide sequence, the plasmid or the vector according to claim 1, as a growth selector for the genetic transformation of the chloroplast genome of microalgae, comprising cultivating in a phosphite-fertilized medium.

6. A method of use of the ptxD nucleotide sequence according to claim 1, as a genetic stabilizer to enhance the expression of recombinant proteins in microalgae cultivations, comprising cultivating in a phosphite-fertilized medium.

7. A chimeric construct comprising:
i) restriction sites at 3′ and 5′ ends;
ii) the nucleotide sequence of a transgene encoding for a protein of interest;
iii) a linker;
iv) a transit peptide;
v) the ptxD nucleotide sequence according to claim 1.

8. The chimeric construct according to claim 7 wherein the linker is a GS linker.

9. The chimeric construct according to claim 7, wherein the transit peptide is a nucleus-encoded, chloroplast-imported precursor protein selected from the group comprising a PreFd element from the preferredoxin protein, the small subunit of RuBisCo multienzyme complex, the pigment-binding Light-Harvesting complex proteins of photosystems or a Calvin Cycle enzyme involved in CO₂ fixation.

10. The chimeric construct according to claim 7, comprising SEQ ID NO:11

11. The chimeric construct according to claim 7 wherein the restriction sites at 3′ and 5′ ends are NcoI and SphI.

12. A method of use of the chimeric construct according to claim 7 as genetic stabilizer and growth selector or the expression of recombinant proteins in microalgae cultivations.

13. The method of use according to claim 12, wherein said microalgae belong to the genus Chlamydomonas or Chlorella.

14. The method of use according to claim 12, wherein said protein of interest is selected from the group consisting of hydrolases, endoglucanases, cellulases, therapeutic peptides, and enzymatic catalysts.

15. A microalgae transformed with the ptxD nucleotide sequence, the plasmid, the vector, the chimeric construct according to claim 1.

16. The microalgae according to claim 15, belonging to the genus Chlamydomonas or Chlorella.

17. A method of use of the microalgae according to claim 15, for the large-scale cultivation and production of recombinant proteins cultivating in a phosphite-fertilized medium.

18. The method of use according to claim 12, wherein said microalgae belong to the species Chlamydomonas reinhardtii, Chlorella vulgaris, Chlorella sorokiniana or Chlorella ohadii.

19. The microalgae according to claim 15, belonging to the species Chlamydomonas reinhardtii, Chlorella vulgaris, Chlorella sorokiniana or Chlorella ohadii.
